SEQUENCE LISTING (36 sequences)

SEQ ID NO: 1 - protein, 153 aa, Artificial Sequence
  Description of Artificial Sequence Synthetic polypeptide
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQL

SEQ ID NO: 2 - protein, 92 aa, Artificial Sequence
  Description of Artificial Sequence Synthetic polypeptide
MHGDTPTLHEYMLDLQPETTQLNDSSEEEDEIDGPAGQAEPDRAHYNIVTFCCKCDSTLRLCVQSTHVDIRTLEDLLMGTLGIVCPICSQKP

SEQ ID NO: 3 - protein, 243 aa, Artificial Sequence
  Description of Artificial Sequence Synthetic polypeptide
MGLKVNVSAIFMAVLLTLQTPTGQIHWGMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLGLSSTSIVYILIAVCLGGLIGIPALICCCRGRCNKKGEQVGMSRPGLKPDLTGTSKSYVRSL

SEQ ID NO: 4 - protein, 185 aa, Artificial Sequence
  Description of Artificial Sequence Synthetic polypeptide
MVPQALLFVPLLVFPLCFGKFPIGSMHGDTPTLHEYMLDLQPETTQLNDSSEEEDEIDGPAGQAEPDRAHYNIVTFCCKCDSTLRLCVQSTHVDIRTLEDLLMGTLGIVCPICSQKPRSYVLLSAGALTALMLIIFLMTCCRRVNRSEPTQHNLRGTGREVSVTPQSGKIISSWESHKSGGETRL

SEQ ID NO: 5 - protein, 9 aa, Artificial Sequence
  Description of Artificial Sequence Synthetic peptide
RAHYNIVTF

SEQ ID NO: 6 - protein, 9 aa, Artificial Sequence
  Description of Artificial Sequence Synthetic peptide
SCVYCKKEL

SEQ ID NO: 7 - protein, 9 aa, Artificial Sequence
  Description of Artificial Sequence Synthetic peptide
RCIICQRPL

SEQ ID NO: 8 - protein, 9 aa, Artificial Sequence
  Description of Artificial Sequence Synthetic peptide
SEYRHYQYS

SEQ ID NO: 9 - protein, 9 aa, Artificial Sequence
  Description of Artificial Sequence Synthetic peptide
ECVYCKQQL

SEQ ID NO: 10 - protein, 9 aa, Artificial Sequence
  Description of Artificial Sequence Synthetic peptide
TDLHCYEQL

SEQ ID NO: 11 - protein, 8 aa, Artificial Sequence
  Description of Artificial Sequence Synthetic peptide
DLYCYEQL

SEQ ID NO: 12 - protein, 8 aa, Artificial Sequence
  Description of Artificial Sequence Synthetic peptide
DLHCYEQL

SEQ ID NO: 13 - protein, 8 aa, Artificial Sequence
  Description of Artificial Sequence Synthetic peptide
DLFCYEQL

SEQ ID NO: 14 - protein, 8 aa, Artificial Sequence
  Description of Artificial Sequence Synthetic peptide
DLLCYEQL

SEQ ID NO: 15 - protein, 8 aa, Artificial Sequence
  Description of Artificial Sequence Synthetic peptide
LQPETTDL

SEQ ID NO: 16 - protein, 8 aa, Artificial Sequence
  Description of Artificial Sequence Synthetic peptide
LQPEATDL

SEQ ID NO: 17 - protein, 8 aa, Artificial Sequence
  Description of Artificial Sequence Synthetic peptide
LEPEATDL

SEQ ID NO: 18 - protein, 8 aa, Artificial Sequence
  Description of Artificial Sequence Synthetic peptide
LYPEPTDL

SEQ ID NO: 19 - protein, 8 aa, Artificial Sequence
  Description of Artificial Sequence Synthetic peptide
LHPEPTDL

SEQ ID NO: 20 - protein, 8 aa, Artificial Sequence
  Description of Artificial Sequence Synthetic peptide
RCLRCQPL

SEQ ID NO: 21 - protein, 8 aa, Artificial Sequence
  Description of Artificial Sequence Synthetic peptide
RCHRCQPL

SEQ ID NO: 22 - protein, 9 aa, Artificial Sequence
  Description of Artificial Sequence Synthetic peptide
RCINCQKPL

SEQ ID NO: 23 - protein, 9 aa, Artificial Sequence
  Description of Artificial Sequence Synthetic peptide
RCIICQKPL

SEQ ID NO: 24 - protein, 9 aa, Artificial Sequence
  Description of Artificial Sequence Synthetic peptide
RCITCQRPL

SEQ ID NO: 25 - protein, 9 aa, Artificial Sequence
  Description of Artificial Sequence Synthetic peptide
RCIICQTPL

SEQ ID NO: 26 - protein, 9 aa, Artificial Sequence
  Description of Artificial Sequence Synthetic peptide
SEYRHYCYS

SEQ ID NO: 27 - protein, 9 aa, Artificial Sequence
  Description of Artificial Sequence Synthetic peptide
SEYRHYNYS

SEQ ID NO: 28 - protein, 9 aa, Artificial Sequence
  Description of Artificial Sequence Synthetic peptide
SEYRWYRYS

SEQ ID NO: 29 - protein, 9 aa, Artificial Sequence
  Description of Artificial Sequence Synthetic peptide
SEFRWYRYS

SEQ ID NO: 30 - protein, 9 aa, Artificial Sequence
  Description of Artificial Sequence Synthetic peptide
TSNYNIVTF

SEQ ID NO: 31 - protein, 9 aa, Artificial Sequence
  Description of Artificial Sequence Synthetic peptide
TSNYNIVTS

SEQ ID NO: 32 - protein, 9 aa, Artificial Sequence
  Description of Artificial Sequence Synthetic peptide
TSNYNIVTY

SEQ ID NO: 33 - protein, 5 aa, Human papillomavirus type 16
CPEEK

SEQ ID NO: 34 - protein, 6 aa, Human papillomavirus type 16
DLYCYE

SEQ ID NO: 35 - protein, 9 aa, Human papillomavirus type 16
TDLYCYEQL

SEQ ID NO: 36 - protein, 9 aa, Human papillomavirus type 16
SEYRHYCYS

(19) United States
(12) Patent Application Publication
(10) Pub. No.: US 2010/0061957 A1
(43) Pub. Date: Mar. 11, 2010

(54) HPV-16-BASED PAPILLOMAVIRUS VACCINES

(75) Inventors: Ronald Rooke, Illkirch Graffenstaden (FR); Stéphane Paul, Saint Just Saint Rambert (FR)

Correspondence Address: BUCHANAN, INGERSOLL & ROONEY PC, POST OFFICE BOX 1404, ALEXANDRIA, VA, 22313-1404, US

(73) Assignee: Transgene S.A., Strasbourg Cedex (FR)

(21) Appl. No.: 12/297,977

(22) PCT Filed: Apr. 17, 2007
(86) PCT No.: PCT/EP07/03368

§ 371 Date: Oct. 21, 2008

(30) Foreign Application Priority Data
Apr. 21, 2006 (EP) 06360014.2

(51) Int. Cl. A61K 38/20; A61K 38/02; A61K 31/7088; A61P 31/12

(52) U.S. Cl. 424/85.2; 514/2; 514/44 R

(57) ABSTRACT

Compositions containing one or more early polypeptide(s) of human papillomavirus (HPV)-16 or a nucleic acid encoding one or more early polypeptide(s) of HPV-16 are useful for preventing or treating an infection or a pathological condition caused by at least one papillomavirus other than HPV-16; the subject compositions are of very special interest in immunotherapy, in particular for preventing or treating HPV persistent infections that might promote cervical intraepithelial neoplasia (CIN) and ultimately cervical cancer.

The present invention relates to the use of a composition comprising one or more early polypeptide(s) of human papillomavirus (HPV)-16 or a nucleic acid encoding one or more early polypeptide(s) of HPV-16 for the manufacture of a medicament for preventing or treating an infection or a pathological condition caused by at least one papillomavirus other than HPV-16. The invention is of very special interest in immunotherapy, in particular in preventing or treating HPV persistent infections possibly leading to cervical intraepithelial neoplasia (CIN) and ultimately to cervical cancer.

Papillomaviruses are small DNA viruses that have been identified in a number of higher organisms including humans (see for example Pfister, 1987, in The papovaviridae: The Papillomaviruses, Salzman and Howley edition, Plenum Press, New York, p 1-38). They are associated with pathological conditions ranging from benign to malignant tumors. In benign tumors, the viral genome is episomal while in malignant tumors, HPV DNA is integrated into the host chromosomes (Stoler, 2000, Int. J. Gynecol. Path. 19, 16-28).

Papillomaviruses possess a double-stranded circular DNA of about 7900 base pairs which is surrounded by a protein capsid. The genome comprises an early (E) region containing the reading frames E1-E7 and a late (L) region. The late region encodes the structural L1 and L2 proteins which form the viral capsid whereas the early genes encode regulatory proteins that are found predominantly in the nucleus. E1 encodes two proteins important in viral genome maintenance and replication. E2 encodes activator and repressor proteins which regulate the viral promoter directing E6 and E7 transcription (Bechtold et al., 2003, J. Virol. 77, 2021-2028). The E4-encoded protein binds and disrupts the cytoplasmic keratin network and may play a role in viral maturation. The role for E5 protein is still controversial and its expression is often lost during viral integration in the host chromosomes. E6 and E7-encoded gene products of cancer-associated HPV genotypes are involved in the oncogenic transformation of infected cells (Kanda et al., 1988, J. Virol. 62, 610-613; Vousden et al., 1988, Oncogene Res. 3, 1-9; Bedell et al., 1987, J. Virol. 61, 3635-3640), which is presumably due to the capacity of these viral proteins to bind cellular tumor suppressor gene products p53 and retinoblastoma (Rb), respectively. The amino acid residues involved in the binding of the native HPV-16 E6 polypeptide to p53 have been clearly defined from residues 118 to 122 (+1 being the first Met residue or from residues 111 to 115 starting from the preferably used second met residue) (Crook et al., 1991, Cell 67, 547-556) and those involved in the binding of the native HPV-16 E7 polypeptide to Rb are located from residues 21 to 26 (Munger et al., 1989, EMBO J. 8, 4099-4105; Heck et al., 1992, Proc. Natl. Acad. Sci. USA 89, 4442-4446).

Currently, over 100 human papillomavirus (HPV) genotypes have been cloned and sequenced (Stoler, 2000, Int. J. Gynecol. Path 19, 16-28). Only 40 HPV genotypes infect the genital mucosa with about 15 of which put women at risk for malignant tumors of the genital tract. More specifically, the two most prevalent genotypes, HPV-16 and HPV-18, are detected in more than 70% of the invasive cervical carcinoma whereas HPV-31, HPV-33 and HPV-45 together accounted for 10% of the cases (Cohen et al., 2005, Science 308, 618-621).

Although cervical screening programs exist, nearly half a million women worldwide are diagnosed with cervical cancers each year and more than 270,000 die according to the data from the International Agency for Research on cancer. The conventional approaches remain surgery and radiotherapy, but new vaccine strategies have been designed for the last 15 years, e.g. peptide-based vaccines (Feltkamp et al., 1993, Eur. J. Immunol. 23, 2242-2249), virus-like particles (VLP) vaccines, DNA vaccines (Osen et al, 2001, Vaccine 19, 4276-4286; Smahel et al., 2001, Virology 281, 231-238) and viral vector vaccines (EP 462,187, Daemen et al., 2000, Gene Ther. 7: 1859-1866; He et al., 2000, Virology 270, 146-161; Borysiewicz et al., 1996, Lancet 347, 1523-1527).

Conceptually, there are two approaches to HPV vaccines, prophylactic and therapeutic. The prophylactic approach seeks to prevent viral infection, i.e. to block virus before it penetrates in the host cells mainly through the induction of neutralizing antibodies. Usually, the prophylactic vaccines target capsid proteins expressed at the virus surface. Most of them rely on recombinantly-produced VLPs of L1 proteins or VLPs mixture of the most prevalent HPV types. Successful phase III clinical trials have been recently reported by Merck and GlaxoSmithKline (GSK) with 100% efficacy at preventing type-specific cervical infections. Cross-protection against oncogenic HPV-31 and HPV-45 genotypes has been described following administration of a mixture of HPV-16 and HPV-18 VLPs (WO 2004/056389). However, the VLP-based preventive vaccines are not expected to induce regression of pathological conditions that develop following HPV infection.

The therapeutic approach seeks to treat established HPV infections and induce regression of HPV-associated precancerous and cancerous pathological conditions mainly through the induction of a cellular immune response. Usually, the therapeutic strategy relies on immunization directed to E6 and/or E7 oncoproteins which are expressed by the HPV-induced tumor cells. So far, immunity provided by the E6 and E7 HPV antigens is considered genotype-specific and the current therapeutic vaccines in clinical or preclinical development focus mainly on the most prevalent oncogenic HPV-16 and to a lesser extend HPV-18.

However, an ideal therapeutic vaccine should permit to provide protection not only against the most prevalent HPV genotypes but also against the other minor HPV genotypes involved in the remaining 30% of cervical cancers. This can be achieved through the development of alternative vaccine candidates directed to each oncogenic HPV genotypes. However, this strategy is likely not to be very attractive in consideration of the cost of clinical and preclinical developments required by regulatory authorities versus the limited number of patients exposed to the minor HPV genotypes.

One may expect that HPV will continue to be a serious global health threat for many years due to the chronic and persistent nature of the infection, its high prevalence and the significant morbidity of HPV-induced cancers. Therefore, there is a need to develop a vaccine offering a broader coverage that is capable of protecting and/or treating against multiple HPV genotypes including in addition to the most prevalent HPV-16 genotype other minor and potentially oncogenic HPV genotypes.

Thus, the present invention represents a significant advance for improving prevention and treatment of papillomavirus infections or papillomavirus-associated pre-malignant and malignant lesions in industrialized countries as well as in developing countries.

This technical problem is solved by the provision of the embodiments as defined in the claims.

Other and further aspects, features and advantages of the present invention will be apparent from the following description of the presently preferred embodiments of the invention. These embodiments are given for the purpose of disclosure.

Accordingly, in a first aspect, the present invention provides the use of a composition comprising one or more early polypeptide(s) of HPV-16 or a nucleic acid encoding one or more early polypeptide(s) of HPV-16 for the manufacture of a medicament for preventing or treating an infection or a pathological condition caused by at least one papillomavirus other than HPV-16.

More particularly, the present invention relates to the use of a composition comprising one or more early polypeptide(s) of HPV-16 or a nucleic acid encoding one or more early polypeptide(s) of HPV-16 for the manufacture of a medicament for treating an infection or a pathological condition caused by at least one human papillomavirus other than HPV-16. The present invention also relates to a method of treating an infection or a pathological condition caused by at least one human papillomavirus other than HPV-16, the method comprising administering to a host organism a composition comprising one or more early polypeptide(s) of HPV-16 or a nucleic acid encoding one or more early polypeptide(s) of HPV-16.

As used herein throughout the entire application, the terms “a” and “an” are used in the sense that they mean “at least one”, “at least a first”, “one or more” or “a plurality” of the referenced compounds or steps, unless the context dictates otherwise. For example, the term “a cell” includes a plurality of cells including a mixture thereof. More specifically, “at least one” and “one or more” means a number which is one or greater than one, with a special preference for one, two or three.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

The term “amino acids” and “residues” are synonyms. These terms refer to natural, unnatural and/or synthetic amino acids, including D or L optical isomers, modified amino acids and amino acid analogs.

The terms “polypeptide”, “peptide” and “protein” are used herein interchangeably to refer to polymers of amino acid residues which comprise nine or more amino acids bonded via peptide bonds. The polymer can be linear, branched or cyclic and may comprise naturally occurring and/or amino acid analogs and it may be interrupted by non-amino acids. As a general indication, if the amino acid polymer is long (e.g. more than 50 amino acid residues), it is preferably referred to as a polypeptide or a protein.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g., cDNA, genomic DNA, plasmids, vectors, viral genomes, isolated DNA, probes, primers and any mixture thereof) or polyribonucleotides (RNA) molecules (e.g., mRNA, antisense RNA) or mixed polyribo-polydeoxyribinucleotides. They encompass single or double-stranded, linear or circular, natural or synthetic polynucleotides. Moreover, a polynucleotide may comprise non-naturally occurring nucleotides, such as methylated nucleotides and nucleotide analogs (see U.S. Pat. No. 5,525,711, U.S. Pat. No. 4,711,955 or EPA 302 175 as examples of modifications) and may be interrupted by non-nucleotide components. If present, modifications to the nucleotide may be imparted before or after polymerization.

As used herein, the term “comprising” when used to define products, compositions and methods, is intended to mean that the products, compositions and methods include the referenced compounds or steps, but not excluding others. “Consisting essentially of” shall mean excluding other compounds or steps of any essential significance. Thus, a composition consisting essentially of the recited compounds would not exclude trace contaminants and pharmaceutically acceptable carriers. “Consisting of” shall mean excluding more than trace elements of other compounds or steps. For example, a polypeptide “consists of” an amino acid sequence when the polypeptide does not contain any amino acids but the recited amino acid sequence. A polypeptide “consists essentially of” an amino acid sequence when such an amino acid sequence is present together with only a few additional amino acid residues, typically from about 1 to about 50 or so additional residues. A polypeptide “comprises” an amino acid sequence when the amino acid sequence is at least part of the final amino acid sequence of the polypeptide. Such a polypeptide can have a few up to several hundred additional amino acids residues. Such additional amino acid residues may play a role in polypeptide trafficking, facilitate polypeptide production or purification; prolong half-life, among other things. The same can be applied for nucleotide sequences.

As used herein, the term “isolated” refers to a protein, polypeptide, peptide or a nucleic acid that is purified or removed from its natural environment. The term “purified” refers to a protein, polypeptide, peptide or a nucleic acid that is separated from at least one other component(s) with which it is naturally associated.

The term “host cell” should be understood broadly without any limitation concerning particular organization in tissue, organ, or isolated cells. Such cells may be of a unique type of cells or a group of different types of cells and encompass cultured cell lines, primary cells and proliferative cells. The term “host organism” refers to a vertebrate, particularly a member of the mammalian species and especially domestic animals, sport animals, and primates including humans.

“HPV” means “human papillomavinis”. Their classification is based on the degree of relatedness of their genomes. More than 100 HPV genotypes have been identified at present time and they have been numbered following the chronological order of their isolation. By convention, two isolates constitute distinct types if they share less than 90% identity in the about 2000 nucleotides long portion of their genome containing the open reading frames E6, E7 and L1. A phylogenetic tree was constructed from the alignment of the available nucleotide sequence (Van Ranst et al., 1992, J. Gen. Virol. 73, 2653; De Villiers et al., 2004, Virology 324, 17-27).

As used herein the term “early polypeptide” refers to an art-recognized non structural protein, selected among the group consisting of E1, E2, E4, E5, E6 and E7 polypeptides with a special preference for E6 and E7. In the context of the invention, the one or more early polypeptide(s) included in the composition or encoded by the nucleic acid included in the composition used according to the invention originate(s) from HPV-16. The term “originate” means be isolated, cloned, derived or related. Thus, in accordance with the present invention, the one or more early HPV-16 polypeptide(s) may originate from a native early HPV-16 polypeptide or a derivative thereof. A “native early HPV-16 polypeptide” refers to a protein, polypeptide or peptide that can be found or isolated from a source in nature, as distinct from being artificially modified or altered by man in the laboratory. Such sources in nature include biological samples (e.g. blood, plasma, sera, vaginal and cervical fluids, tissue sections, biopsies, cytological samples from HPV-16 infected patients), cultured cells, as well as recombinant materials (e.g. HPV-16 virus or genome, genomic or cDNA libraries, plasmids containing fragments of HPV-16 genome, recombinant early HPV-16 polypeptide and the like). Thus the term “native early HPV-16 polypeptide” would include naturally-occurring early HPV-16 polypeptides and fragments thereof. A fragment is preferably of at least 9 amino acid residues and comprises at least one immunogenic epitope. The nucleotide and amino acid sequences of HPV-16 early genes/polypeptides have been described in the literature and are available in specialized data banks, for example in Genbank under accession number NC_{—}01526 and K02718, respectively. However, native early HPV-16 polypeptides are not limited to these exemplary sequences. Indeed the amino acid sequences can vary between different HPV-16 isolates and this natural scope of genetic variation is included within the scope of the invention. Suitable fragments for use in the present invention include the peptides illustrated in the example section, especially the R9F peptide of SEQ ID NO: 5, the E9L peptide of SEQ ID NO: 9, the peptide of HPV-16 E6 polypeptide corresponding to S9S (SEQ ID NO: 8) and the peptide of HPV-16 E7 polypeptide corresponding to T9L (SEQ ID NO: 10). Such peptides can be used independently or in combination (e.g. in fusion).

A derivative of an early HPV-16 polypeptide includes one or more modification(s) with respect to the native HPV-16 early polypeptide, such as those defined below. Modification(s) can be generated by way of mutation and/or addition of chemical moieties (e.g. alkylation, acetylation, amidation, phosphorylation and the like) or labeling moieties. Mutation includes deletion, substitution or addition of one or more amino acid residue(s) or any combination of these possibilities. When several modifications are contemplated, they can concern consecutive residues and/or non consecutive residues. Modification(s) can be made in a number of ways known to those skilled in the art, such as site-directed mutagenesis (e.g. using the Sculptor™ in vitro mutagenesis system of Amersham, Les Ullis, France), PCR mutagenesis and DNA shuffling.

Advantageously, a modified early HPV-16 polypeptide retains a high degree of amino acid sequence identity with the corresponding native early HPV-16 polypeptide over the full length amino acid sequence or a shorter fragment thereof (e.g. of at least 9, 20, 30, 40, 50, 100 amino acids in length), which is greater than 75%, advantageously greater than 80%, desirably greater than 85%, preferably greater than 90%, more preferably greater than 95%, still more preferably greater than 97% (e.g. 100% of sequence identity). The percent identity between two polypeptides is a function of the number of identical positions shared by the sequences, taking into account the number of gaps which need to be introduced for optimal alignment and the length of each gap. Various computer programs and mathematical algorithms are available in the art to determine percentage identities between amino acid sequences such as for example the W2H HUSAR software and the Blast program (e.g. Altschul et al., 1997, Nucleic Acids Res. 25, 3389-3402; Altschul et al., 2005, FEBS J. 272, 5101-5109) available at NCBI.

Desirably, the modified early HPV-16 polypeptide in use according to the invention retains immunogenic activity of the native early HPV-16 polypeptide such as the ability to stimulate a cell-mediated immune response.

In one embodiment, the composition is used for treating HPV infection and/or pathological conditions, especially in the anogenital tract, the skin or the oral cavity, caused by at least one HPV genotype other than HPV-16. In one aspect, the genome of the at least one human papillomavirus share less than 90%, advantageously less than 87% and desirably less than 85% of nucleotide sequence identity with the portion of the HPV-16 genome encoding the E6 or E7 polypeptides but more than 50%, advantageously more than 55% and desirably more than 60% of nucleotide sequence identity with the portion of the HPV-16 genome encoding the E6 or E7 polypeptides. The percent identity between the portions of the HPV genomes is a function of the number of identical positions shared by the two sequences, taking into account the number of gaps which need to be introduced for optimal alignment and the length of each gap. Various computer programs and mathematical algorithms are available in the art to determine percentage identities between nucleotide sequences. Representative examples of such HPV genotypes include without limitation HPV-2, HPV-6, HPV-11, HPV-13, HPV-18, HPV-30, HPV-31, HPV-32, HPV-33, HPV-35, HPV-39, HPV-40, HPV-42, HPV-44, HPV-45, HPV-51, HPV-52, HPV-56, HPV-58, HPV-59, HPV-61, HPV-64 and HPV-68.

Preferably, the at least one human papillomavirus other than HPV-16 is selected among the group consisting of HPV-31, HPV-33, HPV-35, HPV-39, HPV-51, HPV-52, HPV-56, HPV-58, HPV-59 and HPV-68V1, and especially is anyone of HPV-31, HPV-33, HPV-35, HPV-52, and HPV-58 or any possible combination. Representative examples of such combinations include HPV-31 and at least one of HPV-33, HPV-35, HPV-52, and HPV-58; HPV-33 and at least one of HPV-31, HPV-35, HPV-52, and HPV-58; HPV-35 and at least one of HPV-31, HPV-33, HPV-52, and HPV-58; HPV-52 and at least one of HPV-31, HPV-33, HPV-35, and HPV-58; HPV-58 and at least one of HPV-31, HPV-33, HPV-35 and HPV-52. The nucleotide and amino acid sequences of these HPV genotypes have been described in the literature and are available in specialized data banks, as illustrated in Table I.

TABLE I
Genbank accession numbers

| | |
|---|---|
| HPV 18 | X05015 |
| HPV 31 | J04353 |
| HPV 33 | M12732 |
| HPV 35 | NC_001529 |
| HPV 39 | NC_001535 |
| HPV 45 | X74479 |
| HPV 51 | NC_001533 |
| HPV 52 | NC_001592 |
| HPV 56 | X74483 |
| HPV 58 | D90400 |
| HPV 59 | NC_001635 |
| HPV 68 | X67160 |

In another embodiment, the composition used according to the invention comprises or encodes an HPV-16 E6 polypeptide, an HPV-16 E7 polypeptide or both an HPV-16 E6 polypeptide and an HPV-16 E7 polypeptide. Given the observations recalled above on the transforming power of the HPV-16 E6 and E7 polypeptides, modified HPV-16 E6 and/or E7 polypeptides are preferably used which are non-oncogenic variants mutated in the region involved in the interaction with the cellular tumor suppressor gene products p53 and Rb respectively. The present invention encompasses the use of any HPV-16 E6 polypeptide which binding to p53 is altered or at least significantly reduced and/or the use of any HPV-16 E7 polypeptide which binding to Rb is altered or at least significantly reduced (Munger et al., 1989, EMBO J. 8, 4099-4105; Crook et al., 1991, Cell 67, 547-556; Heck et al., 1992, Proc. Natl. Acad. Sci. USA 89, 4442-4446; Phelps et al., 1992, J. Virol. 66, 2148-2427). A non-oncogenic HPV-16 E6 variant which is suitable for the purpose of the present invention is deleted of one or more amino acid residues located from approximately position 118 to approximately position 122 (starting from the first methionine residue of the native HPV-16 E6 polypeptide or from approximately position 111 to approximately position 115 starting from the second methionine residue), with a special preference for the complete deletion of residues 118 to 122 (CPEEK). Most preferred non-oncogenic variant of the HPV-16 E6 polypeptide comprises or alternatively consists essentially of, or alternatively consists of an amino acid sequence which is homologous or identical to the amino acid sequence shown in SEQ ID NO: 1. A non-oncogenic HPV-16 E7 variant which is suitable for the purpose of the present invention is deleted of one or more amino acid residues located from approximately position 21 to approximately position 26 (+1 representing the first amino acid of the native HPV-16 E7 polypeptide, with a special preference for the complete deletion of residues 21 to 26 (DLYCYE). Most preferred non-oncogenic variant of the HPV-16 E7 polypeptide comprises or alternatively consists essentially of, or alternatively consists of an amino acid sequence which is homologous or identical to the amino acid sequence shown in SEQ ID NO: 2.

In a preferred aspect, the one or more HPV-16 early polypeptide(s) in use in the invention is/are further modified so as to improve MHC class I and/or MHC class II presentation, and/or to stimulate anti-HPV immunity. HPV-16 E6 and E7 polypeptides are nuclear proteins and it has been previously shown that membrane presentation permits to improve their therapeutic efficacy (see for example WO99/03885). Thus, it may be advisable to modify at least one of the HPV-16 early polypeptide(s) so as to be anchored to the cell membrane. Membrane anchorage can be easily achieved by incorporating in the HPV-16 early polypeptide a membrane-anchoring sequence and if the native polypeptide lacks it a secretory sequence (i.e. a signal peptide). HPV-16 E6 and/or E7 polypeptide(s) is (are) preferably modified by incorporating a membrane-anchoring sequence and a secretory sequence. Membrane-anchoring and secretory sequences are known in the art. Briefly, secretory sequences are present at the N-terminus of the membrane presented or secreted polypeptides and initiate their passage into the endoplasmic reticulum (ER). They usually comprise 15 to 35 essentially hydrophobic amino acids which are then removed by a specific ER-located endopeptidase to give the mature polypeptide. Membrane-anchoring sequences are usually highly hydrophobic in nature and serve to anchor the polypeptides in the cell membrane (see for example Branden and Tooze, 1991, in Introduction to Protein Structure p. 202-214, NY Garland).

The choice of the membrane-anchoring and secretory sequences which can be used in the context of the present invention is vast. They may be obtained from any membrane-anchored and/or secreted polypeptide comprising it (e.g. cellular or viral polypeptides) such as the rabies glycoprotein, of the HIV virus envelope glycoprotein or of the measles virus F protein or may be synthetic. The membrane anchoring and/or secretory sequences inserted in each of the early HPV-16 polypeptides used according to the invention may have a common or different origin. The preferred site of insertion of the secretory sequence is the N-terminus downstream of the codon for initiation of translation and that of the membrane-anchoring sequence is the C-terminus, for example immediately upstream of the stop codon. Moreover, a linker peptide can be used to connect the secretory sequence to the early HPV-16 polypeptide in use in the invention or to connect the early HPV-16 polypeptide to the membrane anchoring sequence. Linker peptides are known in the art. Typically they contain from 2 to 20 amino acids are include alanine, glycine, proline and/or serine.

The HPV-16 E6 polypeptide in use in the present invention is preferably modified by insertion of the secretory and membrane-anchoring signals of the measles F protein, with a special preference for a polypeptide comprising or alternatively consisting essentially of, or alternatively consisting of an amino acid sequence which is homologous or identical to the amino acid sequence shown in SEQ ID NO: 3. Optionally or in combination, the HPV-16 E7 polypeptide in use in the present invention is preferably modified by insertion of the secretory and membrane-anchoring signals of the rabies glycoprotein, with a special preference for a polypeptide comprising or alternatively consisting essentially of, or alternatively consisting of an amino acid sequence which is homologous or identical to the amino acid sequence shown in SEQ ID NO: 4.

In another and more preferred aspect, the therapeutic efficacy of the composition in use in the invention can also be improved by using one or more immunopotentiator polypeptide(s) or one or more nucleic acid encoding such immunopotentiator polypeptide(s). For example, it may be advantageous to link the HPV-16 early polypeptide(s) to a polypeptide such as calreticulin (Cheng et al., 2001, J. Clin. Invest. 108, 669-678), Mycobacterium tuberculosis heat shock protein 70 (HSP70) (Chen et al., 2000, Cancer Res. 60, 1035-1042), ubiquitin (Rodriguez et al., 1997, J. Virol. 71, 8497-8503) or a bacterial toxin such as the translocation domain of Pseudomonas aeruginosa exotoxin A (ETA(dIII)) (Hung et al., 2001 Cancer Res. 61, 3698-3703). Alternatively, the composition in use in the present invention can further comprise a cytokine or a nucleic acid encoding a cytokine. Suitable cytokines include without limitation interleukin (IL)-2, IL-7, IL-15, IL-18, IL-21 and IFNg, with a special preference for IL-2.

According to another and preferred embodiment, the composition in use according to the invention comprises a nucleic acid encoding one or more HPV-16 early polypeptide(s) as defined above. Preferred is a nucleic acid which encodes at least:
an HPV-16 E6 polypeptide comprising or alternatively consisting essentially of, or alternatively consisting of an amino acid sequence which is homologous or identical to the amino acid sequence shown in SEQ ID NO: 1 or SEQ ID NO:3; and
an HPV-16 E7 polypeptide comprising or alternatively consisting essentially of, or alternatively consisting of an amino acid sequence which is homologous or identical to the amino acid sequence shown in SEQ ID NO: 2 or SEQ ID NO: 4.

If needed, the nucleic acid molecule in use in the invention may be optimized for providing high level expression of the HPV-16 early polypeptide(s) in a particular host cell or organism, e.g. a human host cell or organism. Typically, codon optimisation is performed by replacing one or more “native” (e.g. HPV) codon corresponding to a codon infrequently used in the mammalian host cell by one or more codon encoding the same amino acid which is more frequently used. This can be achieved by conventional mutagenesis or by chemical synthetic techniques (e.g. resulting in a synthetic nucleic acid). It is not necessary to replace all native codons corresponding to infrequently used codons since increased expression can be achieved even with partial replacement. Moreover, some deviations from strict adherence to optimised codon usage may be made to accommodate the introduction of restriction site(s).

Preferably, the HPV-16 early polypeptide-encoding nucleic acid in use in the invention is in a form suitable for its expression in a host cell or organism, which means that the nucleic acid sequence encoding the E6 polypeptide and/or the nucleic acid sequence encoding the E7 polypeptide are placed under the control of one or more regulatory elements necessary for expression in the host cell or organism. As used herein, the term “regulatory element” refers to any sequence that allows, contributes or modulates the expression of the nucleic acid in a given host cell, including replication, duplication, transcription, splicing, translation, stability and/or transport of the nucleic acid or one of its derivative (i.e. mRNA) into the host cell. It will be appreciated by those skilled in the art that the choice of the regulatory elements can depend on factors such as the host cell, the vector and the level of expression desired.

The promoter is of special importance and the present invention encompasses the use of constitutive promoters which direct expression of the nucleic acid in many types of host cells and those which direct expression only in certain host cells or in response to specific events or exogenous factors (e.g. by temperature, nutrient additive, hormone or other ligand). Suitable promoters are widely described in literature and one may cite more specifically viral promoters such as RSV (Rous Sarcoma Virus), SV40 (Simian Virus-40), CMV (Cytomegalo Virus) and MLP (Major Late promoter) promoters. Preferred promoters for use in a poxviral vector include without limitation vaccinia promoters 7.5K, H5R, TK, p28, p11 and K1L, chimeric promoters between early and late poxviral promoters as well as synthetic promoters such as those described in Chakrabarti et al. (1997, Biotechniques 23, 1094-1097), Hammond et al. (1997, J. Virological Methods 66, 135-138) and Kumar and Boyle (1990, Virology 179, 151-158).

Those skilled in the art will appreciate that the regulatory elements controlling the expression of the nucleic acid may further comprise additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), and translation (e.g. tripartite leader sequences, ribosome binding sites, Shine-Dalgamo sequences, etc.) into the host cell or organism.

According to another preferred embodiment, the nucleic acid used according to the present invention is comprised in a vector. The term “vector” as used herein refers to viral as well as non viral (e.g. plasmid DNA) vectors, including extrachromosomal (e.g. episome), multicopy and integrating vectors (i.e. for being incorporated into the host chromosomes). Particularly important in the context of the invention are gene therapy vectors (i.e. which are capable of delivering the nucleic acid to a host organism) as well as expression vectors for use in various expression systems. Suitable non viral vectors include plasmids such as pREP4, pCEP4 (Invitrogene), pCI (Promega), pCDM8 (Seed, 1987, Nature 329, 840), pVAX and pgWiz (Gene Therapy System Inc; Himoudi et al., 2002, J. Virol. 76, 12735-12746). Suitable viral vectors may be derived from a variety of different viruses (e.g. retrovirus, adenovirus, AAV, poxvirus, herpes virus, measle virus, foamy virus and the like). As used herein, the term “viral vector” encompasses vector DNA as well as viral particles generated thereof. Viral vectors can be replication-competent, or can be genetically disabled so as to be replication-defective or replication-impaired. The term “replication-competent” as used herein encompasses replication-selective and conditionally-replicative viral vectors which are engineered to replicate better or selectively in specific host cells (e.g. tumoral cells).

In one aspect, the vector in use in the invention is an adenoviral vector (for a review, see “Adenoviral vectors for gene therapy”, 2002, Ed D. Curiel and J. Douglas, Academic Press). It can be derived from a variety of human or animal sources and any serotype can be employed from the adenovirus serotypes 1 through 51. Particularly preferred are human adenoviruses 2 (Ad2), 5 (Ad5), 6 (Ad6), 11 (Ad11), 24 (Ad24) and 35 (Ad35). Such adenovirus are available from the American Type Culture Collection (ATCC, Rockville, Md.) and have been the subject of numerous publications describing their sequence, organization and methods of producing, allowing the artisan to apply them (see for example U.S. Pat. No. 6,133,028; U.S. Pat. No. 6,110,735; WO 02/40665; WO 00/50573; EP 1016711; Vogels et al., 2003, J. Virol. 77, 8263-8271).

The adenoviral vector in use in the present invention can be replication-competent. Numerous examples of replication-competent adenoviral vectors are readily available to those skill in the art (Hernandez-Alcoceba et al., 2000, Human Gene Ther. 11, 2009-2024; Nemunaitis et al., 2001, Gene Ther. 8, 746-759; Alemany et al., 2000, Nature Biotechnology 18, 723-727). For example, they can be engineered from a wild-type adenovirus genome by deletion in the E1A CR2 domain (e.g. WO00/24408) and/or by replacement of the native E1 and/or E4 promoters with tissue, tumor or cell status-specific promoters (e.g. U.S. Pat. No. 5,998,205, WO99/25860, U.S. Pat. No. 5,698,443, WO00/46355, WO00/15820 and WO01/36650).

Alternatively, the adenoviral vector in use in the invention is replication-defective (see for example WO94/28152; Lusky et al., 1998, J. Virol 72, 2022-2032). Preferred replication-defective adenoviral vectors are E1-defective (e.g. U.S. Pat. No. 6,136,594 and U.S. Pat. No. 6,013,638), with an E1 deletion extending from approximately positions 459 to 3328 or from approximately positions 459 to 3510 (by reference to the sequence of the human adenovirus type 5 disclosed in the GeneBank under the accession number M 73260 and in Chroboczek et al., 1992, Virol. 186, 280-285). The cloning capacity can further be improved by deleting additional portion(s) of the adenoviral genome (all or part of the non essential E3 region or of other essential E2, E4 regions). Insertion of the nucleic acid can be performed through homologous recombination in any location of the adenoviral genome as described in Chartier et al. (1996, J. Virol. 70, 4805-4810). For example, the nucleic acid encoding the HPV-16 E6 polypeptide can be inserted in replacement of the E1 region and the nucleic acid encoding the HPV-16 E7 polypeptide in replacement of the E3 region or vice versa.

In another and preferred aspect, the vector in use in the invention is a poxviral vector (see for example Cox et al. in “Viruses in Human Gene Therapy” Ed J. M. Hos, Carolina Academic Press). It may be obtained from any member of the poxviridae, in particular canarypox, fowlpox and vaccinia virus, the latter being preferred. Suitable vaccinia viruses include without limitation the Copenhagen strain (Goebel et al., 1990, Virol. 179, 247-266 and 517-563; Johnson et al., 1993, Virol. 196, 381-401), the Wyeth strain and the highly attenuated modified Ankara (MVA) strain (Mayr et al., 1975, Infection 3, 6-16). Determination of the complete sequence of the MVA genome and comparison with the Copenhagen genome has allowed the precise identification of seven deletions (I to VII) which occurred in the MVA genome (Antoine et al., 1998, Virology 244, 365-396), any of which can be used to insert the HPV-16 early polypeptide-encoding nucleic acid.

The basic technique for inserting the nucleic acid and associated regulatory elements required for expression in a poxviral genome is described in numerous documents accessible to the man skilled in the art (Paul et al., 2002, Cancer gene Ther. 9, 470-477; Piccini et al., 1987, Methods of Enzymology 153, 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). Usually, one proceed through homologous recombination between overlapping sequences (i.e. flanking the desired insertion site) present both in the viral genome ain a plasmid carrying the nucleic acid to insert.

The nucleic acid is preferably inserted in a nonessential locus of the poxviral genome, in order that the recombinant poxvirus remains viable and infectious. Nonessential regions are non-coding intergenic regions or any gene for which inactivation or deletion does not significantly impair viral growth, replication or infection. One may also envisage insertion in an essential viral locus provided that the defective function be supplied in trans during production of viral particles, for example by using an helper cell line carrying the complementing sequences corresponding to those deleted in the poxviral genome.

When using the Copenhagen vaccinia virus, the HPV-16 early polypeptide-encoding nucleic acid is preferably inserted in the thymidine kinase gene (tk) (Hruby et al., 1983, Proc. Natl. Acad. Sci. USA 80, 3411-3415; Weir et al., 1983, J. Virol. 46, 530-537). However, other insertion sites are also appropriate, e.g. in the hemagglutinin gene (Guo et al., 1989, J. Virol. 63, 4189-4198), in the K1L locus, in the u gene (Zhou et al., 1990, J. Gen. Virol. 71, 2185-2190) or at the left end of the vaccinia virus genome where a variety of spontaneous or engineered deletions have been reported in the literature (Altenburger et al., 1989, Archives Virol. 105, 15-27; Moss et al. 1981, J. Virol. 40, 387-395; Panicali et al., 1981, J. Virol. 37, 1000-1010; Perkus et al, 1989, J. Virol. 63, 3829-3836; Perkus et al, 1990, Virol. 179, 276-286; Perkus et al, 1991, Virol. 180, 406-410).

When using MVA, the HPV-16 early polypeptide-encoding nucleic acid can be inserted in anyone of the identified deletions I to VII as well as in the D4R locus, but insertion in deletion II or III is preferred (Meyer et al., 1991, J. Gen. Virol. 72, 1031-1038; Sutter et al., 1994, Vaccine 12, 1032-1040).

When using fowlpox virus, although insertion within the thymidine kinase gene may be considered, the HPV-16 early polypeptide-encoding nucleic acid is preferably introduced in the intergenic region situated between ORFs 7 and 9 (see for example EP 314 569 and U.S. Pat. No. 5,180,675).

As described above, the composition in use in the invention can further comprise a cytokine-expressing nucleic acid. It may be carried by the vector encoding the one or more HPV-16 early polypeptide(s) or by an independent vector which can be of the same or a different origin.

A preferred embodiment of the invention is directed to the use of a composition comprising a MVA vector encoding the HPV-16 E6 polypeptide placed under the 7.5K promoter, the HPV-16 E7 polypeptide placed under the 7.5K promoter and the human IL-2 gene placed under the control of the H5R promoter. Preferably, nucleic acids encoding the HPV-16 E6 polypeptide, the HPV-16 E7 polypeptide and the human IL-2 are inserted in deletion III of the MVA genome.

In addition, the composition in use in the invention may include one or more stabilizing substance(s), such as lipids (e.g. cationic lipids, liposomes, lipids as described in WO98/44143), nuclease inhibitors, hydrogel, hyaluronidase (WO98/53853), collagenase, cationic polymers, polysaccharides, chelating agents (EP890362), in order to preserve its degradation within the animal/human body and/or improve transfection/infection of the vector into the host cell or organism. Such substances may be used alone or in combination (e.g. cationic and neutral lipids).

Infectious viral particles comprising the above-described nucleic acid or vectors can be produced by routine process. An exemplary process comprises the steps of:

(a) introducing the viral vector into a suitable cell line,

(b) culturing said cell line under suitable conditions so as to allow the production of said infectious viral particle,

(c) recovering the produced infectious viral particle from the culture of said cell line, and

(d) optionally purifying said recovered infectious viral particle.

When the viral vector is defective, the infectious particles are usually produced in a complementation cell line or via the use of a helper virus, which supplies in trans the non functional viral genes. For example, suitable cell lines for complementing E1-deleted adenoviral vectors include the 293 cells (Graham et al., 1997, J. Gen. Virol. 36, 59-72) as well as the PER-C6 cells (Fallaux et al., 1998, Human Gene Ther. 9, 1909-1917). Cells appropriate for propagating poxvirus vectors are avian cells, and most preferably primary chicken embryo fibroblasts (CEF) prepared from chicken embryos obtained from fertilized eggs.

The infectious viral particles may be recovered from the culture supernatant or from the cells after lysis (e.g. by chemical means, freezing/thawing, osmotic shock, mecanic shock, sonication and the like). The viral particles can be isolated by consecutive rounds of plaque purification and then purified using the techniques of the art (chromatographic methods, ultracentrifugation on cesium chloride or sucrose gradient).

The present invention also encompasses the use of vectors or viral particles that have been modified to allow preferential targeting to a particular target host cell (see for example Wickam et al., 1997, J. Virol. 71, 8221-8229; Arnberg et al., 1997, Virol. 227, 239-244; Michael et al., 1995, Gene Therapy 2, 660-668; WO94/10323; WO02/96939 and EP 1 146 125). A characteristic feature of targeted vectors and viral particles is the presence at their surface of a ligand capable of recognizing and binding to a cellular and surface-exposed component such as a cell-specific marker (e.g. an HPV-infected cell), a tissue-specific marker (e.g. a cervix-specific marker), as well as a viral (e.g. HPV) antigen. Examples of suitable ligands include antibodies or fragments thereof directed to an HPV antigenic domain. The ligand is usually genetically inserted in a polypeptide present on the surface of the virus (e.g. adenoviral fiber, penton, pIX or vaccinia p14 gene product).

The composition in use the present invention can be produced by any suitable method, for example, by standard direct peptide synthesizing techniques (e.g. Bodanszky, 1984 in Principles of peptide synthesis, Springer-Verlag) and by recombinant DNA technology in appropriate host cells. For example, the nucleic acid coding for the HPV-16 E6 and E7 early polypeptides can be isolated directly from HPV-containing cells (e.g. Caski cells), cDNA and genomic libraries, viral genomes or any prior art vector known to include it, by conventional molecular biology or PCR techniques. If needed, it can further be modified by routine mutagenesis techniques. Alternatively, the nucleic acid in use in the invention can also be generated by chemical synthesis in automatised process (e.g. assembled from overlapping synthetic oligonucleotides as described for example in Edge, 1981, Nature 292, 756; Nambair et al., 1984, Science 223, 1299; Jay et al., 1984, J. Biol. Chem. 259, 6311). Those skilled in the art are knowledgeable in the numerous expression systems available for producing the HPV-16 early polypeptides in appropriate host cells and of the methods for introducing a vector or an infectious viral particle into a host cell.

A preferred use of the composition according to the invention is for treating a variety of diseases and pathological conditions, especially those associated with an HPV infection caused by at least one of the HPV genotypes listed above. Although the invention also encompasses prophylaxy, it is especially useful for therapy, e.g. for treating HPV persistent infection, precancerous as well as cancerous conditions which may develop in HPV-infected patients. Examples of HPV-associated cancerous conditions include cervical carcinoma, anal carcinoma and oral cancer. HPV-associated precancerous conditions extend from low grade to high grade lesions including cervical intra-epithelial neoplasia (CIN) of grade 1, 2 or 3.

Preferably, upon administration into a host organism according to the modalities described herein, the composition of the invention provides a therapeutic benefit to the treated host organism. The therapeutic benefit can be evidenced by a number of ways as compared to before treatment, for instance at a population level by a decrease of frequency of HPV infections, by a delay in the development of a pathological condition typically associated with HPV infection (e.g. delay in the development of CIN lesions or cervical cancers) or at the individual level by a decrease of HPV viremia, and/or an inhibition of viral gene expression (e.g. a decrease HPV E6 or E7-expressing RNAs) and/or by an improvement of the clinical outcome (e.g. stabilization, partial or total regression of an HPV-associated lesion) and/or by a stimulation of the immune system resulting in the development of an enhanced anti-HPV response whether humoral or cellular or both (e.g. production of anti-HPV antibodies and/or T cell-mediated immunity) and/or by an improved response of the host organism to conventional therapies. For example, the composition used according to the invention provides a benefit when its administration to HPV positive women is followed by (i) a negative HPV detection following one or more positive detections, (ii) a regression of high grade CIN2/3 lesions to low grade CIN 1 or (iii) a stabilization or regression of an invasive cervical carcinoma. A regular follow up of the patients after treatment is recommended over a minimum of 6 months.

The presence of HPV can be determined in biological fluid (e.g. a vaginal or cervical fluids, blood, serum, plasma), gynaecologic samples collected using conventional cervical sampling device, tissue sections, and biopsies. A variety of methods are available to those skilled in the art to evaluate the presence of HPV DNA and RNA in a sample, such as LiPA system (WO99/14377; Labo Biomedical products, Netherlands), Pre Tect HPV Proofer (N or Chip AS, Norway), Hybrid Capture II system (Digene Corp, USA), Thin Prep System (Cytyc Corporate; Marlborough, Mass.) and PCR/RT-PCR systems. Suitable primers are known to the skilled person or can be easily synthesized on the basis of the nucleotide sequence of the HPV genotype of interest. One may also proceed by immunogenicity assays (e.g. ELISA) using suitable antibodies. Regression or stabilization of an HPV-induced lesion can be determined by measuring the actual size of the lesion over a period of time. Direct observation (e.g. colposcopy), radiologic imaging methods, immunologic imaging methods or ultrason may be used to estimate the size of the lesion over time. In addition, a variety of in vitro methods may be used in order to predict stabilization or regression of an HPV-associated lesion in a host organism, such as cytological and histological analysis to estimate the presence of atypical cells. Stimulation of an anti-HPV immune response may be estimated a number of routine techniques such as those described below in connection with the use of the composition for inducing or stimulating an immune response.

Suitably, the composition of the invention further comprises a pharmaceutically acceptable vehicle. As used herein, a “pharmaceutically acceptable vehicle” is intended to include any and all carriers, solvents, diluents, excipients, adjuvants, dispersion media, coatings, antibacterial and antifungal agents, and absorption delaying agents, and the like, compatible with pharmaceutical administration. The pharmaceutically acceptable vehicle(s) included in the composition must also permit to preserve its stability under the conditions of manufacture and long-term storage (i.e. at least one month) at freezing (e.g. −70° C., −20° C.), refrigerated (e.g. 4° C.) or ambient temperature (e.g. 20° C.) or in a lyophilized state.

The composition in use in the invention is suitably buffered in order to be appropriate for human use at a physiological or slightly basic pH (e.g. between about pH 7 to about pH 9). Suitable buffers include without limitation phosphate buffer (e.g. PBS), bicarbonate buffer and/or Tris buffer.

In addition it may comprise a diluent appropriate for human or animal use. Such a diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see for example the most current edition of Remington: The Science and Practice of Pharmacy, A. Gennaro, Lippincott, Williams & Wilkins).

The composition may also contain other pharmaceutically acceptable excipients for providing desirable pharmaceutical or pharmacodynamic properties, including for example modifying or maintaining osmolarity, viscosity, clarity, colour, sterility, stability, rate of dissolution of the formulation, modifying or maintaining release or absorption into an the human or animal organism, promoting transport across the blood barrier or penetration in a particular organ (e.g. liver). Suitable excipients include amino acids.

In addition, the composition may be used in combination with conventional adjuvant(s) suitable for systemic or mucosal application in humans.

The composition may be administered to the host organism by a variety of modes of administration, including systemic, topical and localized administration. Suitable administration routes include without limitation subcutaneous, intradermal, intramuscular, intravenous, intraperitoneal, intratumoral, intravascular, and intraarterial injection. Injections can be made with conventional syringes and needles, or any other appropriate devices available in the art. Alternatively the composition may be administered via a mucosal route, such as the oral/alimentary, nasal, intratracheal, intrapulmonary, intravaginal or intra-rectal route. Topical administration can also be performed using transdermal means (e.g. patch and the like). In the context of the invention, intramuscular and subcutaneous administrations constitute the preferred routes. The administration may take place in a single dose or a dose repeated one or several times after a certain time interval varying from a day to a year. Desirably, intervals are a matter of one week to one month.

The appropriate dosage can be adapted as a function of various parameters, in particular the mode of administration; the composition employed; the age, health, and weight of the host organism; the nature and extent of symptoms; kind of concurrent treatment; the frequency of treatment; and/or the need for prevention or therapy. Further refinement of the calculations necessary to determine the appropriate dosage is routinely made by a practitioner, in the light of the relevant circumstances. For general guidance, suitable dosage for a vaccinia-containing composition varies from about 10⁴ to 10⁹ pfu (plaque forming units), desirably from about 10⁵ and 10⁸ pfu whereas adenovirus-comprising composition varies from about 10⁵ to 10¹³ iu (infectious units), desirably from about 10⁷ and 10¹¹ iu. A composition based on vector plasmids may be administered in doses of between 10 μg and 20 mg, advantageously between 100 μg and 2 mg. A protein composition may be administered in doses of between 10 ng and 20 mg, with a special preference for a dosage from about 0.1 μg to about 2 mg per kg body weight.

In a preferred embodiment, the composition in use in the invention comprises the above-described MVA vector and is administered in three doses of 5×10⁵ pfu to 5×10⁷ pfu by subcutaneous route at weekly intervals.

If desired, the use of the invention can be carried out in conjunction with one or more conventional therapeutic modalities (e.g. radiation, chemotherapy and/or surgery). Multiple therapeutic approaches provide the patient with a broader based intervention. In one embodiment, the method of the invention can be preceded or preferably followed by a surgical excision of the HPV-associated lesion (e.g. conisation). In another embodiment, it can be preceded or followed by radiotherapy (e.g. gamma radiation). Those skilled in the art can readily formulate appropriate radiation therapy protocols and parameters which can be used (see for example Perez and Brady, 1992, Principles and Practice of Radiation Oncology, 2nd Ed. JB Lippincott Co; using appropriate adaptations and modifications as will be readily apparent to those skilled in the field). In still another embodiment, the method or use of the invention is associated to chemotherapy with one or more drugs which are conventionally used for treating or preventing HPV infections, HPV-associated pathologic conditions.

In another embodiment, the use of the invention is carried out according to a prime boost therapeutic modality which comprises sequential administration of one or more priming composition(s) and one or more boosting composition(s). Typically, the priming and the boosting compositions use different vehicles which comprise or encode at least an immunogenic domain in common. The priming composition is initially administered to the host organism and the boosting composition is subsequently administered after a time period varying from one day to twelve months. Moreover, the priming and boosting compositions can be administered at the same site or at alternative sites by the same route or by different routes of administration. For example, a priming composition based on HPV-16 early polypeptide(s) can be administered by a mucosal route whereas a boosting composition based on nucleic acid vector is preferably injected, e.g. subcutaneous injection for a MVA vector, intramuscular injection for a DNA plasmid and for an adenoviral vector.

The present invention also pertains to the use of a composition comprising one or more early polypeptide(s) of HPV-16 or a nucleic acid encoding one or more early polypeptide(s) of HPV-16 for inducing or stimulating an immune response against at least one human papillomavirus other than HPV-16. The invention also relates to a method of inducing or stimulating in a mammal an immune response against at least one human papillomavirus other than HPV-16, the method comprising administering to the mammal a composition comprising one or more early polypeptide(s) of HPV-16 or a nucleic acid encoding one or more early polypeptide(s) of HPV-16. The immune response is preferably a cellular immune response directed to an HPV early polypeptide, with a preference for a CD4+, a CD8+ or both a CD4+ and a CD8+-mediated immune response.

The ability to induce or stimulate an anti-HPV immune response upon administration in an animal or human organism can be evaluated either in vitro or in vivo using a variety of assays which are standard in the art. For a general description of techniques available to evaluate the onset and stimulation of an immune response, see for example Coligan et al. (1992 and 1994, Current Protocols in Immunology; ed J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFNg-producing cells by ELIspot), by determination of the activation status of immune effector cells (e.g. T cell proliferation assays by a classical [³H] thymidine uptake), by assaying for antigen-specific T lymphocytes in a sensitized subject (e.g. peptide-specific lysis in a cytotoxicity assay), by lymphocyte mediated anti-tumor cytolytic activity determined for example, by a ⁵¹Cr release assay. The ability to stimulate a humoral response may be determined by antibody binding and/or competition in binding (see for example Harlow, 1989, Antibodies, Cold Spring Harbor Press) or by in vitro generation of tumor specific antibody-mediated inhibition of cell growth (Gazit et al., 1992, Cancer Immunol. Immunother 35, 135-144). The method of the invention can also be further validated in animal models challenged with an appropriate tumor-inducing agent (e.g. HPV-16 E6 and E7-expressing TC1 cells) to determine anti-tumor activity, reflecting an induction or a stimulation of an anti-HPV immune response.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### LEGENDS OF FIGURES

FIG. 1 illustrates MVATG8042

FIG. 2 illustrates E7/E6-specific IFNg ELISPOT assay (means/group). Groups are defined by the immunogen used, either MNA N33 (white) or MVATG8042 (grey). Results are represented as the median of immunized group.

The following examples serve to illustrate the present invention.

### EXAMPLES

### Materials and Methods

Viruses

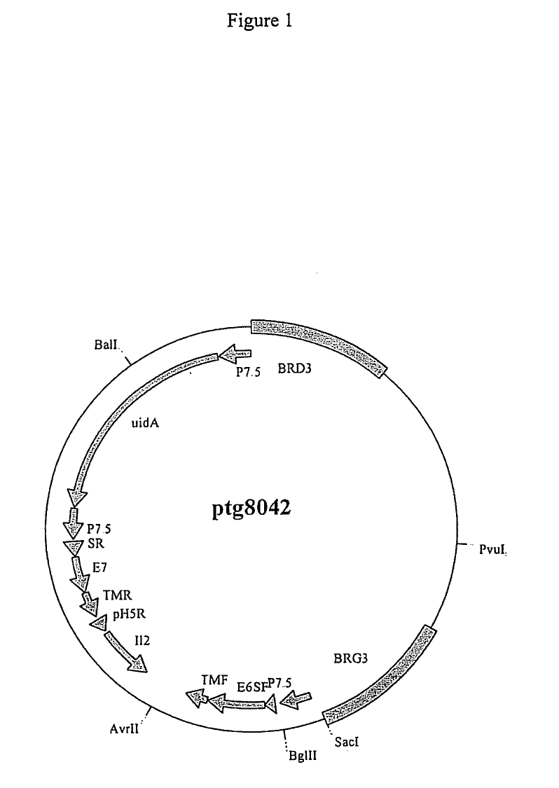

MVATG8042 (FIG. 1) is a recombinant MVA virus expressing membrane anchored and non-oncogenic variants of HPV-16 E6 and E7 polypeptides (E6*TMF and E7*TMR) as well as human IL-2. MVATG8042 is described in WO99/03885 and U.S. Pat. No. 6,884,786. The HPV-16 gene sequences are both placed under the control of the p7.5K promoter whereas the IL-2 gene is driven by the H5R promoter and all are inserted into the region of excision III of the MVA genome.

Virus particles of MVATG8042 are produced in CEF cells according to conventional techniques. Virus stocks were maintained at −80° C. until the day of injection. The viral suspension was rapidly thawed, and diluted before administration in TG0008 buffer containing Tris-HCl 10 mM pH8, saccharose 5% (w/v), and 50 mM NaCl, in order to obtain the viral dose of 5×10⁷ pfu in a 100 μl volume.

Animal Model

SPF healthy female C57Bl/6 mice were obtained from Charles River (Les Oncins, France). The animals were housed in a single, exclusive room air-conditioned to provide a minimum of 11 air changes per hour. The temperature and relative humidity ranges were within 18° C. and 22° C. and 40 to 70% respectively. Lighting was controlled automatically to give a cycle of 12 hours of light and 12 hours of darkness. Throughout the study the animals had access ad libitum to sterilized diet type RM1 (SDS, France). Sterile water was provided ad libitum via bottles.

7-week-old C57Bl/6 female mice were immunized subcutaneously 3 times at day 0, 7 and 14 with 5×10⁷ pfu of MVATGN33 or MVATG8042. Subcutaneous injections were performed each time in a different location of the right flank of the animals. Spleens were taken at day 21 after the last immunization. Fresh spleen cells were prepared using conventional techniques in the art.

A 96-well nitrocellulose plate was coated with 3 μg/ml monoclonal rat anti-mouse IFNg antibody (Clone R4-6A2; Pharmingen, Cat Number 551216, 100 μl/well) in Sodium Carbonate Buffer. The plates were incubated overnight at 4° C. or 1 h at 37° C. Plates were washed three times with DMEM 10% FCS and saturated 2 hours at 37° C. with 100 μl DMEM 10% FCS/well. Splenocytes were plated at a concentration of 10⁶ cells/100 μl. IL-2 was added to the wells at a concentration of 6 U/50 μl/well (R&D Systems; 10 ng/ml). Concanavalin A was used as positive control (5 μg/ml).

All the peptides were synthesized by Neosystem. Each peptide was dissolved in DMSO at 10 mg/ml and store at 4° C. Peptides were used at a concentration of 5 μg/ml. The plates were incubated 48 hours at 37° C., in 5% CO₂.

The plate was washed one time with PBS 1× and 5 times with PBS-Tween 0.05%. Biotinylated Anti-mouse IFNg (clone XMG1.2, Pharmingen) was added at the concentration of 0.3 μg/100 μl/well and incubated 2 hours at room temperature under slow agitation. The plate was washed 5 times with PBS-Tween 0.05%. Extravidin AKP (Sigma, St. Louis, Mo.) diluted at 1/5000 in PBS-Tween0.05%-FCS1% was also added to the wells (100 μl/well). The plate was incubated 45 minutes at room temperature and then washed 5 times with PBS-Tween 0.05%. IFNg secretion was revealed with Biorad Kit. 100 μl substrate (NBT+BCIP) was added per well and plate was left at room temperature for 0.5 hour. The plate was washed with water and put to dry overnight at room temperature. Spots were counted using a dissecting microscope.

RESULTS

The E6 and E7 amino acid sequences from different HPV genotypes were aligned using HUSAR multiple alignment program (CLUSTAL) (https://genius.embnet.dkfz-heidelberg.de/menu/cgi-bin/w2h/w2h.start).

H2^{b}-restricted peptides (Db or Kb restricted) were identified using the BIMAS peptide binding software available on the Internet (http://bimas.dcrt.nih.gov/molbio/hla_bind/). The R9F peptide present in the HPV16-E7 protein (RAHYNIVTF: SEQ ID NO: 5) was used as a reference peptide. It has been described in the art as capable of being recognized by E7-specific CTL and was identified in the BIMAS data with a binding score of 6. The amino acid sequence of non HPV-16 E6 and E7 peptides identified with scores equal or above this value were aligned with that of the corresponding peptide in HPV-16 E6 and E7. Peptides showing one or two amino acid differences with respect to the amino acid sequence HPV-16 E6 and E7 polypeptides were elected for this cross-reactivity analysis. Six peptides were tested:

SCVYCKKEL (HPV56 E6 Db): S9L PEPTIDE (SEQ ID NO: 6)

RCIICQRPL (HPV33, E6 HPV 58 E6 Db): R9L, PEPTIDE (SEQ ID NO: 7)

SEYRHYQYS (HPV52, E6 Kb): S9S PEPTIDE (SEQ ID NO: 8)

ECVYCKQQL (HPV16, E6 Db): E9L PEPTIDE (SEQ ID NO: 9)

TDLHCYEQL (HPV31, E7 Kb): T9L PEPTIDE (SEQ ID NO: 10); and

RAHYNIVTF (HPV16, E7 Db): PEPTIDE R9F (SEQ ID NO: 5) as positive control

Irrelevant peptide: as negative control

For example, the peptide T9L has been identified with a binding score of 20 in HPV-31 and HPV-52 E7 polypeptides. It shows one amino acid difference with respect to the corresponding HPV-16 E7 peptide (TDLYCYEQL). The S9S peptide has been identified with a binding score of 15.8 in HPV-52 E6 polypeptide and it shows one amino acid difference with respect to the corresponding HPV-16 E6 peptide (SEYRHYCYS).

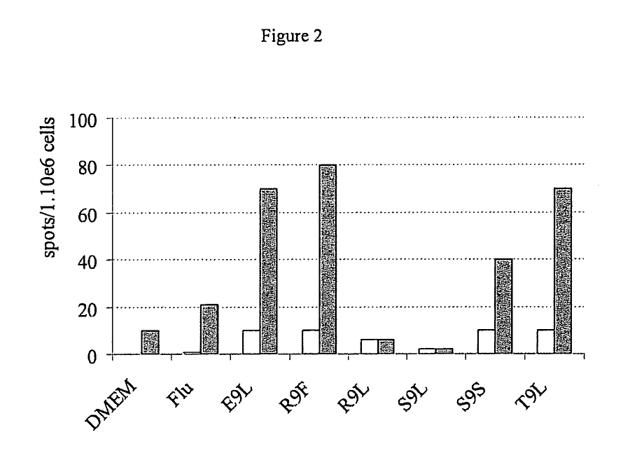

Cross reactivity was assessed by IFNg ELISPOT assay on splenocytes obtained from mice immunized with MVATG8042 as described in Materials and Methods. The results are shown in FIG. 2. Immunization of mice with non-recombinant MVATGN33 does not induce any Th1 response (production of IFNg below the basal level). On the other hand, immunization with MVATG8042 induces a multi-epitopes Th1 response in mice. As expected, the culture of immunized splenocytes with R9F peptide stimulates production of IFNg whereas the addition of an irrelevant Flu peptide in the splenocytes culture has no significant effect (production of IFNg at the basal level). However, surprisingly, other peptides than the known E7H2^{b}-restricted R9F peptide are recognized by CTL such as the S9S, E9L, and T9L peptides. Moreover, T-cell stimulation with HPV31- or HPV52-specific peptides seems as potent as that generated with the CTL-recognized R9F E7-peptide. These observations have been made on the basis of MHC class I molecules. It could not be excluded that other genotypes could be presented by MHC class II molecules.

Furthermore, it should be noted that the sequence of the HPV31- and HPV-52-specific T9L peptide matches with the sequence of the corresponding peptide of HPV 33, 35, and 58 sequences with the exception of one amino acid.

Cross Stimulation Experiment

A cross-stimulation experiment was performed in order to determine if splenocytes from MVATG8042 immunized mice could be stimulated by peptides specific to other HPV genotypes. To limit the number of peptides to be tested, regions of either E6 or E7 protein with high probability of association with MHC class I molecules (Db and Kb) were identified using the Bimass software. A series of peptides were tested which exhibit one, two or three amino acid differences with respect to the corresponding peptide from HPV-16 E6 or E7 (see Table 1). All the peptides were synthesized by Neosystem (France) at the immunograde level. Each peptide was dissolved in DMSO at 10 mg/ml and stored at 4° C. The number of IFNγ-producing cells per 10⁶ splenocytes was evaluated in the peptide-stimulated splenocytes taken from naïve or MVATG8042-vaccinated animals.

TABLE 1
List of tested peptides

| | | | Genotype | |
|---|---|---|---|---|
| | | | showing 100% | |
| | | | homology to the | |
| Peptide | | | peptide | |
| denomination | Sequence | Protein | sequence | SEQ ID |
| D8L-1 | DLYCYEQL | E7 | 16, 33, 35 | SEQ ID NO: 11 |
| D8L-2 | DLHCYEQL | E7 | 31, 52 | SEQ ID NO: 12 |
| D8L-3 | DLFCYEQL | E7 | 58 | SEQ ID NO: 13 |
| D8L-4 | DLLCYEQL | E7 | 18, 45 | SEQ ID NO: 14 |
| E9L | ECVYCKQQL | E7 | 16 | SEQ ID NO: 9 |
| L8L-1 | LQPETTDL | E7 | 16, 52 | SEQ ID NO: 15 |
| L8L-2 | LQPEATDL | E7 | 31 | SEQ ID NO: 16 |
| L8L-3 | LEPEATDL | E7 | 35 | SEQ ID NO: 17 |
| L8L-4 | LYPEPTDL | E7 | 33 | SEQ ID NO: 18 |
| L8L-5 | LHPEPTDL | E7 | 58 | SEQ ID NO: 19 |
| R9F | RAHYNIVTF | E7 | 16 | SEQ ID NO: 5 |
| R8L-1 | RCLRCQPL | E6 | 18, 45 | SEQ ID NO: 20 |
| R8L-2 | RCHRCQPL | E6 | 51 | SEQ ID NO: 21 |
| R9L | RCIICQRPL | E6 | 33, 58 | SEQ ID NO: 7 |
| R9L-2 | RCINCQKPL | E6 | 16 | SEQ ID NO: 22 |
| R9L-3 | RCIICQKPL | E6 | 35 | SEQ ID NO: 23 |
| R9L-4 | RCITCQRPL | E6 | 31 | SEQ ID NO: 24 |
| R9L-5 | RCIICQTPL | E6 | 52 | SEQ ID NO: 25 |
| S9L-3 | SCVYCKKEL | E6 | 56 | SEQ ID NO: 6 |
| S9S | SEYRHYQYS | E6 | 52 | SEQ ID NO: 8 |
| S9S-2 | SEYRHYCYS | E6 | 16 | SEQ ID NO: 26 |
| S9S-3 | SEYRHYNYS | E6 | 33, 58 | SEQ ID NO: 27 |
| S9S-4 | SEYRWYRYS | E6 | 52 | SEQ ID NO: 28 |
| S9S-5 | SEFRWYRYS | E6 | 31 | SEQ ID NO: 29 |
| T9F | TSNYNIVTF | E7 | 31 | SEQ ID NO: 30 |
| T9L | TDLHCYEQL | E7 | 31 | SEQ ID NO: 10 |
| T9S | TSNYNIVTS | E7 | 35 | SEQ ID NO: 31 |
| T9Y | TSNYNIVTY | E7 | 52 | SEQ ID NO: 32 |

Briefly, C57Bl/6 female mice were immunized three times subcutaneously with 5×10⁷ pfu of MVATGN33 (one mouse as negative control) or MVATG8042 (three mice). Subcutaneous injections were performed each time in a different location of the right flank of the animals. Spleens were taken at day 21 after the last immunization and fresh spleen cells were prepared using a Cell Strainer (BD Falcon). Cross-stimulation of the various peptides with respect to the HPV-16-immunized splenocytes was evaluated by Elispot using the Mabtech AB mouse IFNγ ELISPOT^{PLUS} kit or mouse IL-4 ELISPOT^{PLUS} kit (Mabtech, France) according to the manufacturer's instructions. The plate was washed with water and put to dry overnight at room temperature. Spots were counted using the Elispot reader Bioreader 4000 Pro-X (BIOSYS-Gmbh; Serlabo France). For each peptide, the number of spots represents the mean of duplicate from which was subtracted the mean of duplicate of background. Background values are the number of spots obtained with a Kb-restricted irrelevant peptide. Peptides from non-HPV-16 genotypes were considered to be able to cross-stimulate splenocytes from MVATG8042-immunized animals when at least 30 spots were seen and that the number was twice the value seen for the same peptide in the naïve animal.

Peptide restimulation in naïve non injected animals did not stimulate any significant cellular immune response. In marked contrast and as expected, a high number of spots was observed after restimulation of the splenocytes obtained from MVATG8042-immunized mice with the known E7H2^{b}-restricted R9F peptide. However, surprisingly, other peptides than the R9F peptide are recognized by CTL especially the T9L (HPV-31), T9F (HPV-31), T9S(HPV-35) and T9Y (HPV-52) peptides.

Altogether, these data provides a positive trend that vaccination with HPV-16 E6 and/or E7 polypeptides or expressing vectors (e.g. MVATG8042) could also be efficacious for treating infections with the minor and oncogenic HPV of genotypes 31, 33, 35, and 52.

1.-25. (canceled)

26. A regime or regimen for preventing or treating an infection or a pathological condition caused by at least one papillomavirus other than HPV-16, comprising administering to a subject in need of such treatment, a thus effective amount of a composition which comprises one or more early polypeptide(s) of HPV-16 or a nucleic acid encoding one or more early polypeptide(s) of HPV-16.

27. A regime or regimen for treating an infection or a pathological condition caused by at least one human papillomavirus other than HPV-16, comprising administering to a subject in need of such treatment, a thus effective amount of a composition which comprises one or more early polypeptide(s) of HPV-16 or a nucleic acid encoding one or more early polypeptide(s) of HPV-16.

28. A regime or regimen for inducing an immune response against at least one human papillomavirus other than HPV-16, comprising administering to a subject in need of such treatment, a thus effective amount of a composition which comprises one or more early polypeptide(s) of HPV-16 or a nucleic acid encoding one or more early polypeptide(s) of HPV-16.

29. The regime or regimen as defined by claim 26, wherein said at least one human papillomavirus other than HPV-16 is selected from the group consisting of HPV-31, HPV-33, HPV-35, HPV-39, HPV-51, HPV-52, HPV-56, HPV-58, HPV-59 and HPV-68V1.

30. The regime or regimen as defined by claim 26, wherein said one or more HPV-16 early polypeptide(s) comprise an E6 polypeptide, an E7 polypeptide or both an E6 polypeptide and an E7 polypeptide.

31. The regime or regimen as defined by claim 30, wherein said HPV-16 E6 and/or E7 polypeptide(s) is (are) non-oncogenic variant(s).

32. The regime or regimen as defined by claim 31, wherein said non-oncogenic variant of the HPV-16 E6 polypeptide comprises an amino acid sequence which is homologous or identical to the amino acid sequence shown in SEQ ID NO: 1.

33. The regime or regimen as defined by claim 31, wherein said non-oncogenic variant of the HPV-16 E7 polypeptide comprises an amino acid sequence which is homologous or identical to the amino acid sequence shown in SEQ ID NO: 2.

34. The regime or regimen as defined by claim 30, wherein said HPV-16 E6 and/or E7 polypeptide(s) is (are) modified so as to be anchored to the cell membrane by incorporating a membrane-anchoring sequence and a secretory sequence.

35. The regime or regimen as defined by claim 34, wherein said membrane-anchoring sequence and/or secretory sequence are obtained from the rabies glycoprotein, the HIV virus envelope glycoprotein or the measles virus F protein.

36. The regime or regimen as defined by claim 35, wherein said HPV-16 E6 polypeptide comprises an amino acid sequence which is homologous or identical to the amino acid sequence shown in SEQ ID NO: 3.

37. The regime or regimen as defined by claim 35, wherein said HPV-16 E7 polypeptide comprises an amino acid sequence which is homologous or identical to the amino acid sequence shown in SEQ ID NO: 4.

38. The regime or regimen as defined by claim 26, wherein said composition further comprises a cytokine or a nucleic acid encoding a cytokine.

39. The regime or regimen as defined by claim 38, wherein said cytokine comprises IL-2.

40. The regime or regimen as defined by claim 26, wherein said nucleic acid encoding one or more HPV-16 early polypeptide(s) comprises a vector therefor.

41. The regime or regimen as defined by claim 40, wherein said viral vector comprises a vaccinia vector.

42. The regime or regimen as defined by claim 41, wherein said vaccinia vector comprises a MVA vector.

43. The regime or regimen as defined by claim 42, wherein said MVA vector comprises a nucleic acid encoding the HPV-16 E6 polypeptide placed under the 7.5K promoter, a nucleic acid encoding the HPV-16 E7 polypeptide placed under the 7.5K promoter and the human IL-2 gene placed under the control of the H5R promoter.

44. The regime or regimen as defined by claim 43, wherein said nucleic acids encoding said HPV-16 E6 polypeptide, said HPV-16 E7 polypeptide and said human IL-2 gene are inserted in deletion III of the MVA genome.

45. The regime or regimen as defined by claim 26, wherein, wherein said pathological condition is HPV persistent infection, a precancerous or a cancerous condition.

46. The regime or regimen as defined by claim 45, wherein said HPV-associated cancerous condition is a cervical carcinoma, an anal carcinoma or an oral cancer.

47. The regime or regimen as defined by claim 45, wherein said HPV-associated precancerous condition comprises a cervical intra-epithelial neoplasia (CIN) of grade 1, 2 or 3.

48. The regime or regimen as defined by claim 26, comprising administering said composition by subcutaneous or intramuscular route.

49. The regime or regimen as defined by claim 45, wherein said composition is administered at dose(s) comprising from 5×10⁵ pfu to 5×10⁷ pfu of vaccinia vector.

50. The regimen as defined by claim 49, wherein said composition comprises a MVA vector and is administered in three doses of 5×10⁵ pfu to 5×10⁷ pfu by subcutaneous route at weekly intervals.
